(19) United States
(12) Patent Application Publication
(10) Pub. No.: US 2013/0218027 A1
(43) Pub. Date: Aug. 22, 2013

(54) IMAGING DEVICE AND METHODS OF USING THE SAME

(71) Applicant: BOSTON SCIENTIFIC SCIMED, INC., (US)

(72) Inventor: Sandra Nagale, Lowell, MA (US)

(73) Assignee: BOSTON SCIENTIFIC SCIMED, INC., Maple Grove, MN (US)

(21) Appl. No.: 13/770,564

(22) Filed: Feb. 19, 2013

Related U.S. Application Data
(60) Provisional application No. 61/601,835, filed on Feb. 22, 2012.

(51) Int. Cl. A61B 5/00

(52) U.S. Cl. CPC A61B 5/0084
USPC 600/478

(57) ABSTRACT

In one embodiment, a medical device includes a first optical fiber, a second optical fiber, a third optical fiber, and a fourth optical fiber. The first optical fiber is operatively coupled to a first electromagnetic radiation source and is configured to transmit electromagnetic radiation to bodily tissue. The second optical fiber is configured to receive electromagnetic radiation from the first electromagnetic radiation source scattered by the bodily tissue. The third optical fiber is operatively coupled to a second electromagnetic radiation source and is configured to transmit electromagnetic radiation to bodily tissue. The second electromagnetic radiation source is different than the first electromagnetic radiation source. The fourth optical fiber is configured to receive electromagnetic radiation from the second electromagnetic radiation source scattered by the bodily tissue.

### CROSS-REFERENCE TO RELATED APPLICATION

This application is a Nonprovisional of, and claims priority to, U.S. Patent Application No. 61/601,835, filed Feb. 22, 2012, entitled “IMAGING DEVICE AND METHODS OF USING THE SAME”, which is incorporated by reference herein in its entirety.

### TECHNICAL FIELD

This disclosure relates generally to medical devices and more particularly to imaging devices and methods of using imaging devices.

### BACKGROUND

A variety of medical devices, such as imaging devices, are used to detect abnormalities within a body of a patient. For example, imagining devices may be used to observe and detect bodily tissue that is unhealthy or diseased. Some imaging devices may be used to detect cancerous or precancerous tissue within a body of a patent.

Some known imaging devices and methods provide for a low-invasive endoscopic diagnosis of cancerous or precancerous lesions within a body of a patient. Some of the known imaging devices provide for (1) a survey of a wide area of the body of the patient, such as an organ of a patient, (2) imaging penetration within the area where neoplastic changes occur, and (3) the capability to detect differences in optical properties of healthy bodily tissue and unhealthy bodily tissue, such as cancerous or precancerous bodily tissue. Some known imaging devices, however, may not provide sufficiently high diagnostic accuracy. For example, some known imaging devices may provide a 90 to 96% diagnostic accuracy. Thus, some of the known imaging devices and methods may not provide sufficiently high sensitivities or specificities to replace current non-imaging cancer surveillance protocols, such as biopsies.

Accordingly, there is a need for an imaging device and method that would provide a low-invasive process for identifying unhealthy tissue, such as cancerous or precancerous bodily tissue within a body of a patient. Additionally, there is a need for an imaging device that provides high diagnostic accuracy.

### SUMMARY

In one embodiment, a medical device includes a first transmitting member, a second transmitting member, a third transmitting member, and a fourth transmitting member. The first transmitting member is operatively coupled to a first electromagnetic radiation source and is configured to transmit electromagnetic radiation to bodily tissue. The second transmitting member is configured to receive electromagnetic radiation from the first electromagnetic radiation source scattered by the bodily tissue. The third transmitting member is operatively coupled to a second electromagnetic radiation source and is configured to transmit electromagnetic radiation to bodily tissue. The second electromagnetic radiation source is different than the first electromagnetic radiation source. The fourth transmitting member is configured to receive electromagnetic radiation from the second electromagnetic radiation source scattered by the bodily tissue.

In another embodiment, method includes inserting a medical device into a body of a patient such that the medical device is disposed adjacent bodily tissue, delivering electromagnetic radiation of a first electromagnetic radiation source to the bodily tissue, delivering electromagnetic radiation of a second electromagnetic radiation source to the bodily tissue, the second electromagnetic radiation source being different than the first electromagnetic radiation source, and removing the medical device from the body of the patient.

In another embodiment, a method includes receiving an amount of electromagnetic radiation of a first electromagnetic radiation source as scattered by bodily tissue, receiving an amount of electromagnetic radiation of a second electromagnetic radiation source as scattered by the bodily tissue, forming a fingerprint of the bodily tissue that includes data associated with the amount of electromagnetic radiation received from the first electromagnetic radiation source and data associated with the amount of electromagnetic radiation received from the second electromagnetic radiation source; and providing a diagnosis of the bodily tissue.

### BRIEF DESCRIPTION OF THE DRAWINGS

FIG. 1 is a schematic illustration of medical device according to an embodiment of the invention.

FIG. 2 is a perspective view of a distal portion of a medical device according to an embodiment of the invention.

FIG. 3 is a cross-sectional view of the distal portion of the medical device of FIG. 2.

FIG. 4 is a cross-sectional view of a distal portion of a medical device according to an embodiment of the invention.

FIG. 5 is a cross-sectional view of a distal portion of a medical device according to an embodiment of the invention.

FIG. 6 illustrates a sample fingerprint of bodily tissue.

FIGS. 7 and 8 are flow charts of methods according to embodiments of the invention.

### DETAILED DESCRIPTION

The device and methods described herein are generally directed to bodily imaging devices. For example, the devices may be configured to be placed within a body of a patient and provide an image or data associated with bodily tissue of the patient. For example, in some embodiments, the medical device is configured to be inserted into a bodily lumen of a patient (such as an esophagus or rectum of a patient) and provide imaging or data associated with the bodily tissue of the patient surrounding or within the bodily lumen. In other embodiments, the medical device may be used in other locations within the body of the patient.

The terms proximal and distal are used to describe some of the embodiments herein. The term proximal is used to refer to the portion or end of the device that is closest to the operator or the physician (such as a portion of the device that extends from the body of the patient). The term distal is used to refer to the portion or end of the device that is furthest from the operator or the physician (such as the portion of the device that extends into the body of the patient).

FIG. 1 is a schematic illustration of a medical device 100 according to an embodiment of the invention. In some embodiments, the medical device 100 is configured to be at least partially disposed within a body of a patient and detect unhealthy bodily tissue of the patient. In some embodiments, the medical device 100 is configured to perform a plurality of measurements to determine whether the bodily tissue of the patient is healthy or unhealthy.

In some embodiments, the medical device 100 incorporates a plurality of imaging modalities to analyze the bodily tissue. For example, in some embodiments, the medical device 100 incorporates a light scattering analysis (such as an angle-resolved low coherence interferometry (aLCI), elastic scattering spectroscopy (ESS), or any other light scattering technology) and a Raman scattering analysis. The combination of the plurality of analysis or modalities may provide diagnostic results that provide a high sensitivity and a high selectivity. For example, one analysis alone may provide a high sensitivity and another analysis alone may provide a high selectivity and the combined analysis may provide both, a high sensitivity and a high selectivity.

In some embodiments, more than two modalities may be used to analyze bodily tissue. For example, in some embodiments, three or more modalities may be used to analyze the bodily tissue. In some embodiments, one modality used to analyze bodily tissue may be a non-optical modality. For example, ultrasound or an ultrasonic modality may be used to analyze the bodily tissue. In some embodiments, two or more than two optical modalities may be used together with a non-optical modality.

The medical device 100 includes a probe portion 102 and a control portion 104. The probe portion 102 is configured to be disposed at least partially within a body of a patient when the device 100 is in use. The control portion 104 is configured to be disposed outside of the body of the patient and is configured to control, or includes portions or components that are configured to control, the medical device 100. As described in more detail below, the control portion 104 includes a first electromagnetic radiation source 150 (such as a first light source), a second electromagnetic radiation source 170 (such as a second light source), a first electromagnetic radiation detector 160 (such as a first spectrometer), and a second electromagnetic radiation detector 180 (such as second spectrometer), and a computing device, such as a computer, 190.

The probe portion 102 of the medical device 100 includes a first transmitting member 110, a second transmitting member 120, a third transmitting member 130, and a fourth transmitting member 140. In some embodiments, the transmitting members 110, 120, 130, 140 are configured to be disposed within a body of a patient such that a portion of each of the transmitting members 110, 120, 130, and 140 are disposed within the body of the patient and a portion of each of the transmitting members 110, 120, 130, and 140 are disposed outside of the body of the patient. For example, in some embodiments, the transmitting members 110, 120, 130, and 140 are configured to be placed in at least partially within a bodily lumen (such as an esophagus or rectum) of the patient. In some embodiments, the medical device 100 may be used for, but not limited to, gastrointestinal applications, urological applications, and gynecological applications.

The probe portion 102 of the medical device 100 may have any geometrical shape. In some embodiments, the geometrical shape of the probe portion 102 of the medical device 100 may help facilitate the accurate transmission or receipt of electromagnetic radiation. For example, the probe portion 102 of the medical device 100 may have a bulbous portion or may include a sidewall that includes a substantially flat portion. In other embodiments, the probe portion 102 of the medical device may include a pinpoint or narrow head portion.

In some embodiments, the probe portion 102 includes additional transmitting members to accommodate the various modalities of analysis. For example, the probe portion 102 may include additional, or more than 4 transmitting members, in embodiments that provide for more than two modalities of analysis.

In some embodiments, the medical device 100 includes an approximation or anchoring member. The approximation or anchoring member may help facilitate the accurate transmission or receipt of electromagnetic radiation. For example, in some embodiments, the device 100 includes one or more suction ports that are configured to help anchor or approximate the medical device 100 to the bodily tissue. The suction ports may allow the medical device 100 to achieve closer access to the bodily tissue. In other embodiments, the medical device 100 includes a distance control mechanism that is configured to help control the distance between the medical device 100 and the bodily tissue that is being observed with the device.

The transmitting members 110, 120, 130, and 140 may be any type transmitting device such as an electromagnetic radiation or light transmitting device. For example, the transmitting members 110, 120, 130, and 140 may each be any type of type of device that is configured to transmit light from one location (such as a location outside of a body of a patient) to another location (such as a location within the body of the patient). For example, in some embodiments, the transmitting members 110, 120, 130, and 140 are each a light transmitting fiber (e.g., single mode fiber, multi-mode fiber, a fiber with various types of cladding). In other embodiments, the transmitting members 110, 120, 130, and 140 are each a plurality or group of light transmitting fibers. For example, the transmitting members maybe a plurality of optical fibers that are bundled together. In some embodiments, the transmitting members 110, 120, 130, and 140 may be disposed in any geometry (such as a symmetric or an asymmetric geometry) within the device 100. In yet further embodiments, the transmitting members 110, 120, 130, and 140 are other types of devices or materials that are configured to transmit electromagnetic radiation, light, or light waves from one location to another location.

The first transmitting member 110 includes a first end portion 112 and a second end portion 114. In some embodiments, the first end portion 112 is configured to be disposed outside of a body of a patient while the second end portion 114 is configured to be disposed within the body of the patient. The first end portion 112 is operatively coupled to the first electromagnetic radiation source 150. As described in further detail below, in some embodiments, the electromagnetic radiation source 150 is configured to generate and direct light (such as light of varying wavelengths) into the transmitting member 110. In some embodiments, the first end portion 112 may also be operatively coupled to (or configured to be operatively coupled to) the second electromagnetic radiation source 170 and configured to transmit the radiation or light of the second electromagnetic source 170 to a location within the body of the patient.

The transmitting member 110 is configured to receive the electromagnetic radiation or light from the electromagnetic radiation source 150 and transmit the radiation or light to a location within the body of the patient. More specifically, the transmitting member 110 is configured to transmit the radiation or light to a location within the body of the patient and direct the radiation or light towards or into bodily tissue of the patient. For example, in some embodiments, the first transmitting member 110 is configured to transmit the radiation or light of the first electromagnetic radiation source 150 such that the radiation or light escapes or exits the transmitting member 110 from the distal or second end portion 114 of the transmitting member 110.

In some embodiments, the first transmitting member 110 is flexible and includes a curved portion towards the distal or second end portion 114 that is configured to direct the electromagnetic radiation or light of the first electromagnetic radiation source 150 towards bodily tissue of the patient. In other embodiments, the first transmitting member 110 or a portion of the first transmitting member 110, such as the distal or second end portion 114, is configured to direct the electromagnetic radiation or light of the first electromagnetic radiation source 150 towards a reflecting member (not shown). In such embodiments, the reflecting member is configured to direct the electromagnetic radiation or light towards bodily tissue of the patient.

In some embodiments, the first electromagnetic radiation source 150 is configured to produce or generate waves, such as light waves, and direct the light waves to the first transmitting member 110. In some embodiments, the first electromagnetic radiation source 150 is a lamp or other type of light generation device. In some embodiments, the first electromagnetic radiation source 150 is configured to produce or generate light within a range of specific wavelengths. For example, in some embodiments, the first electromagnetic radiation source is a pulsed xenon arc lamp and is configured to produce light with wavelengths between 320 to 920 nm. In other embodiments, the first electromagnetic radiation source 150 is a different type of bulb and is configured to produce light with a different range of wavelengths.

In some embodiments, the first electromagnetic radiation source 150 is a wide band source. In other embodiments, the first electromagnetic radiation source 150 is a narrow band source. In some embodiments, a filter is disposed between the first electromagnetic radiation source 150 and the first transmitting member 110 to limit the band of wavelengths of radiation that is delivered to the transmitting member 110 and to the body of the patient.

The second transmitting member 120 includes a first end portion 122 and a second end portion 124. The second transmitting member 120 is configured to transmit electromagnetic radiation or light from its second end portion 124 to its first end portion 122. The second transmitting member 120 is configured to receive electromagnetic radiation, light, or light waves from within the body of the patient and transmit such electromagnetic radiation, light, or light waves from a location within the body of the patient to a location outside of the body of the patient. For example, in some embodiments, the second transmitting member 120 is configured to receive electromagnetic radiation or light of the first electromagnetic radiation source 150 that has been delivered to bodily tissue of the patient via the first transmitting member 110 and scattered (such as by reflection or by emitting) by the bodily tissue. Said another way, electromagnetic radiation, light, or light waves of the first source are transmitted from the first electromagnetic radiation source 150 to the bodily tissue via the first transmitting member 110. The radiation, light, or light waves or some portion of the light or light waves may be scattered or reflected by the bodily tissue. In other embodiments, the bodily tissue may scatter electromagnetic radiation by emitting electromagnetic radiation in response to being exposed to the electromagnetic radiation of the first electromagnetic radiation source 150. The radiation, light, or light waves that are scattered by the bodily tissue is received by the distal end portion 124 of the second transmitting member 120. In some embodiments, the second transmitting member 120 is configured to receive and transmit any type or a plurality of types of energy or light waves from a location within the body of the patient to a location outside of the body of the patient.

The first end portion 122 of the second transmitting member 120 is operatively coupled to the first electromagnetic radiation detector 160. Once the radiation, light, or light waves are received by the second end portion 124 of the second transmitting member 120, the radiation, light, or light waves are transmitted to the first end portion 122 of the second transmitting member 120 and delivered to the first electromagnetic radiation detector 160.

The first electromagnetic radiation detector 160 is configured to receive the radiation, light, or the light waves and provide data for analysis. For example, in some embodiments, the first electromagnetic radiation detector 160 is a spectrometer and is configured to output intensity data or a spectrum of the radiation, light, or light waves received by the second transmitting member 120. In some embodiments, the first electromagnetic radiation detector 160 is configured to deliver the output to the computing device 190, which is configured to analyze the output. In some embodiments, the computing device 190 is configured to analyze the output according to a first modality, such as an angle-resolved low coherence interferometry (aLCI) or another modality.

The third transmitting member 130 includes a first end portion 132 and a second end portion 134. In some embodiments, the first end portion 132 is configured to be disposed outside of a body of a patient while the second end portion 134 is configured to be disposed within the body of the patient. The first end portion 132 is operatively coupled to the second electromagnetic radiation source 170. As described in further detail below, the second electromagnetic radiation source 170 is configured to generate and direct electromagnetic radiation, such as light (such as light of varying wavelengths) into the third transmitting member 130. In some embodiments, the second electromagnetic radiation source 170 is different than the first electromagnetic radiation source 150. For example, in some embodiments, the first electromagnetic radiation source 150 is of a first type and the second electromagnetic radiation source 170 is of a second, different type.

The third transmitting member 130 is configured to receive the electromagnetic radiation or light from the second electromagnetic radiation source 170 and transmit the radiation or light to a location within the body of the patient. More specifically, the third transmitting member 130 is configured to transmit the radiation or light to a location within the body of the patient and direct the radiation or light towards or into bodily tissue of the patient. For example, in some embodiments, the third transmitting member 130 is configured to transmit the radiation or light of the second electromagnetic radiation source 170 such that the radiation or light escapes or exits the third transmitting member 130 from the distal or second end portion 134 of the third transmitting member 130.

In some embodiments, the third transmitting member 130 is flexible and includes a curved portion towards the distal or second end portion 134 that is configured to direct the electromagnetic radiation or light of the second electromagnetic radiation source 170 towards bodily tissue of the patient. In other embodiments, third transmitting member 130 or a portion of the third transmitting member 130, such as the distal or second end portion 134, is configured to direct the electromagnetic radiation or light of the second electromagnetic radiation source 170 towards a reflecting member. In such embodiments, the reflecting member is configured to direct the radiation or light towards bodily tissue of the patient.

In some embodiments, the second electromagnetic radiation source 170 is configured to produce or generate electromagnetic radiation or light waves and direct the radiation or light waves to the third transmitting member 130. In some embodiments, the second electromagnetic radiation source 170 is a diode laser or another type of light generation device. In some embodiments, the second electromagnetic radiation source 170 is configured to produce or generate electromagnetic radiation or light within a range of specific wavelengths. For example, in some embodiments, the second electromagnetic radiation source 170 is configured to produce radiation or light with wavelengths between about 5,000 and 13,000 nm. In other embodiments, the second electromagnetic radiation source 170 is a configured to produce radiation or light with a different range of wavelengths.

The fourth transmitting member 140 includes a first end portion 142 and a second end portion 144. The fourth transmitting member 140 is configured to transmit electromagnetic radiation or light from its second end portion 144 to its first end portion 142. The fourth transmitting member 140 is configured to receive radiation, light, or light waves from within the body of the patient and transmit such radiation, light, or light waves from a location within the body of the patient to a location outside of the body of the patient. For example, in some embodiments, the fourth transmitting member 140 is configured to receive radiation, or light, of the second electromagnetic radiation source 170 that has been delivered to bodily tissue of the patient via the third transmitting member 130 and scattered (such as reflected or emitted) by the bodily tissue. Said another way, electromagnetic radiation, light, or light waves of the first source are transmitted from the second electromagnetic radiation source 170 to the bodily tissue via the third transmitting member 130. The radiation, light, or light waves or some portion of the light or light waves may be scattered (either reflected or emitted) by the bodily tissue. The radiation, light, or light waves that are scattered by the bodily tissue is received by the distal end portion 144 of the fourth transmitting member 140.

The first end portion 142 of the fourth transmitting member 140 is operatively coupled to the second electromagnetic radiation detector 180 (such as a spectrometer, a camera, or other type of detector). Once the radiation, light, or light waves are received by the second end portion 144 of the fourth transmitting member 140, the radiation, light, or light waves are transmitted to the first end portion 142 of the fourth transmitting member 140 and delivered to the second electromagnetic radiation detector 180.

The second electromagnetic radiation detector 180 is configured to receive the radiation, light, or the light waves from the fourth transmitting member 140 and provide data for analysis. For example, in some embodiments, the second electromagnetic radiation detector 180 is configured to output intensity data or a spectrum of the light or light waves received by the fourth transmitting member 140. In some embodiments, the second electromagnetic radiation detector 180 is configured to deliver the output to the computing device 190, which is configured to analyze the output. In some embodiments, the computing device 190 is configured to analyze the output according to a second modality, different than the first modality. For example, in some embodiments, according to a Raman scattering approach.

In some embodiments, the electromagnetic radiation sources 150 and 170 are configured to scan ranges of wavelengths. For example, the electromagnetic radiation sources 150 and 170 may be configured to emit a first wavelength at a first time and second wavelength at a second time, and so on until the wavelengths of the entire range of that particular electromagnetic radiation source have been emitted. In some embodiments, the computer or computing device 190 is operatively coupled to the electromagnetic radiation sources 150 and 170 and configured to control the electromagnetic radiation sources 150 and 170 (such as the generation of the light waves by the electromagnetic radiation sources 150 and 170).

In some embodiments, the probe portion 102 of the device 100, including the transmitting members 110, 120, 130, and 140 may be moved within the body of the patient to probe or analyze different bodily tissue within the body of the patient. For example, the probe portion 102 of the device 100 may be configure to be inserted into the body of the patient and moved to different locations or depths within the body of the patient (such as along the bodily lumen, such as the esophagus or the rectum, at different locations). Additionally, in some embodiments, the probe portion 102 is configured to be rotated within the body of the patient, such as within the bodily lumen, to probe or analyze different bodily tissue or all of the bodily tissue that forms the bodily lumen. In other embodiments, the transmitting members 110, 120, 130, and 140 are rotatably coupled to or within the probe portion 102 of the medical device 100. In such embodiments, the transmitting members 110, 120, 130, and 140 may be rotated or moved with respect to the probe portion 102 to analyze different bodily tissue.

In some embodiments, the first transmitting member 110, the second transmitting member 120, the third transmitting member 130, and the fourth transmitting member 140 are coupled together. For example, in some embodiments, the transmitting members may be inserted into the body of the patient as a unit or together. Additionally, in some embodiments, the probe portion 102 of the device 100 includes a housing 103 that is configured to house or receive at least a portion of each of the light transmitters. For example, the housing 103 may define a lumen or a channel that is configured to house or receive at least a portion of each of the light transmitters.

In some embodiments, as will be described in more detail below, the output or analysis of the first modality and the output or analysis of the second modality may be combined to provide a diagnosis of the bodily tissue that has been observed. For example, in some embodiments, the combined analysis may provide a diagnosis or indication that the bodily tissue is healthy or a diagnosis or indication that the bodily tissue is unhealthy or diseased. Specifically, the combined analysis may provide an indication that the observed tissue is cancerous or pre-cancerous.

In some embodiments, the combined analysis may provide a highly accurate diagnosis of the bodily tissue. For example, in some embodiments, the combined analysis may provide a diagnosis that has a high sensitivity and a high selectivity. Specifically, in some embodiments, the combined analysis may provide a diagnosis that has 99% sensitivity and 98% selectivity.

In some embodiments, as described in more detail below, the output or analysis of the first modality and the output of the second modality may be combined to create a fingerprint of the observed bodily tissue. The fingerprint of the observed bodily tissue may then be compared to other fingerprints of known healthy tissues and of known diseased tissues. The comparison of the fingerprint of the observed tissue with the other fingerprints of other tissues of known states, may provide the diagnosis of the observed tissue.

While the medical device 100 is illustrated and described as including two transmitting member and two receiving members, any number of such members may be used. For example, a single transmitting member may be used to transmit two different types of electromagnetic radiation from a location outside of the body to a location within the body of the patient. In other embodiments, more than one transmitting member (such as a bundle of transmitting members) may be used to transmit a single type of electromagnetic radiation from a location within the body to a location outside of the body of the patient.

When the medical device 100 is illustrated and described as transmitting two types of signals within two different ranges of wavelengths, in other embodiments, the medical device 100 is configured to deliver a different number of ranges or wavelengths to the body of the patient. For example, in some embodiments, the device 100 may be configured to provide and receive a third type of signal or range of wavelength to the body of the patient.

In some embodiments, the electromagnetic radiation that is emitted by the medical device 100 may be controlled. For example, the amplitude, the frequency, the waveform shape, the pulse rate, the polarization, the frequency, or the phase may be controlled. In some embodiments, the electromagnetic radiation that is received is monitored for a change in any or all of the controlled parameters.

In some embodiments, a single transmitter or a single electromagnetic radiation generator may be used to deliver light from a plurality of different ranges of wavelengths. For example, the generator could be controlled to deliver two radiations of two different ranges. The generator could generate and deliver the radiation of the different ranges in intervals or super positioned. In such an embodiment, the receiver may be configured to split the received signal into portions of the radiation from the different ranges.

In some embodiments, the electromagnetic radiation generator and the electromagnetic radiation detector are the same device. In other words, a single device may perform both functions of generating and detecting the electromagnetic radiation. For example, in some embodiments, the device may alternate between generating the electromagnetic radiation and receiving the electromagnetic radiation.

In some embodiments, the electromagnetic radiation may be transmitted through a material to help facilitate the transmission of the radiation. For example, the radiation may be transmitted through a material such as a gel, fluid or polymer to facilitate the transmission of the radiation.

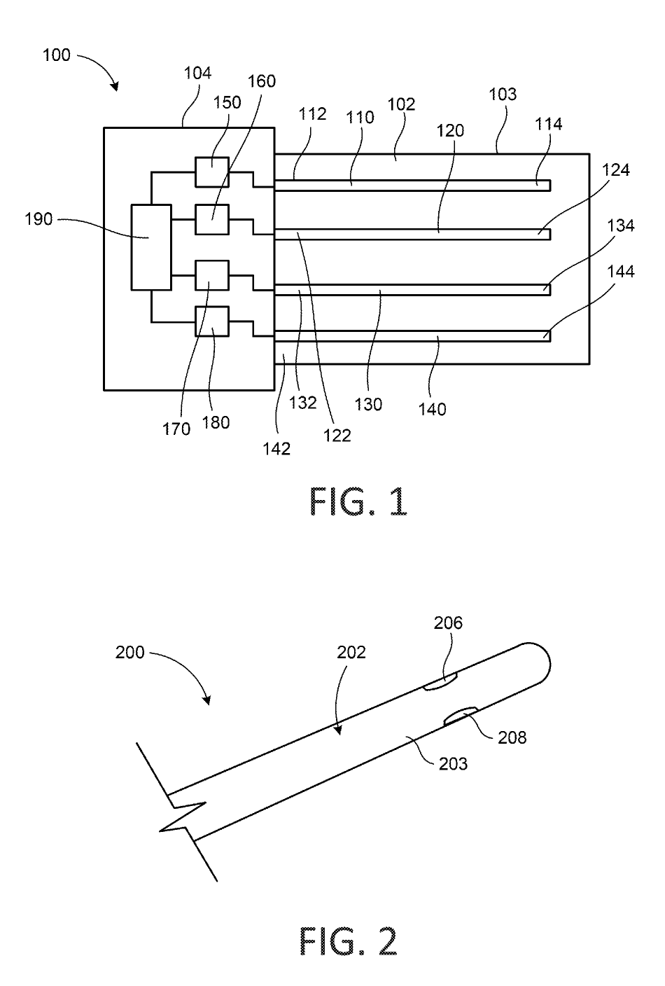

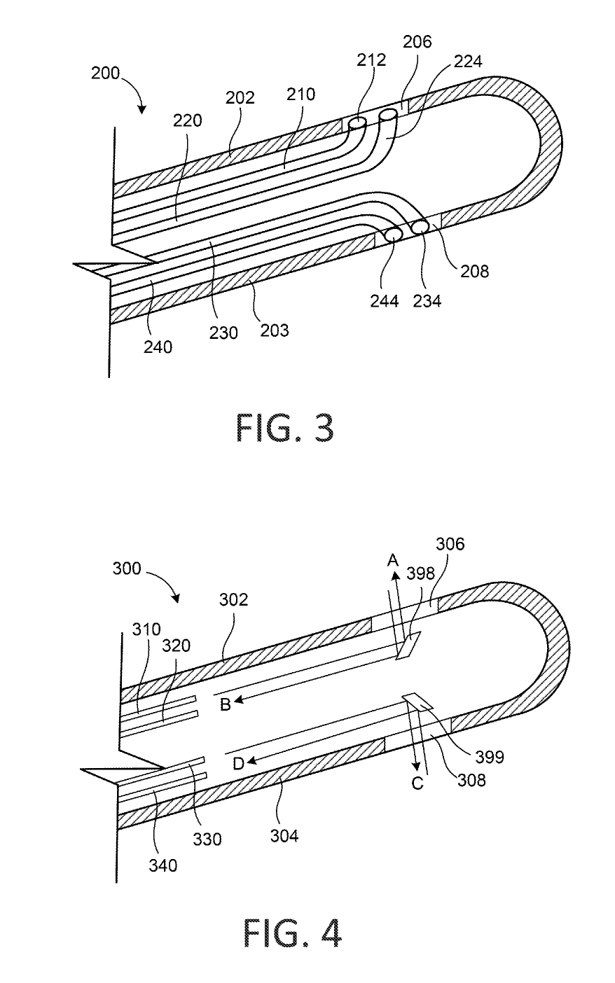

FIG. 2 is a perspective view of a probe portion 202 of a medical device 200. FIG. 3 is a cross-sectional view of a portion of the probe portion 202 of FIG. 2. The probe portion 202 (or at least a portion probe portion 202) is configured to be disposed or inserted into a body of a patient. For example, the probe portion 202 may be inserted into a bodily lumen of a patient. The probe portion 202 may be inserted into the body such that it is disposed adjacent bodily tissue that is to be observed. The probe portion 202 may be inserted moved shallower or deeper into the body of the patient depending on the tissue that is to be observed. Also, in some embodiments, the probe portion 202 may be moved from one location within the body of the patient to another location within the body of the patient to scan the bodily tissue or observe different bodily tissue portions. Also, in some embodiments, the probe portion 202 may be rotated such that the tissue of all portions of the bodily lumen may be observed.

The probe portion 202 includes a housing 203. The housing 203 defines a lumen or cavity that is configured to house at least a portion of a first transmitting member 210, a second transmitting member 220, a third transmitting member 230, and a fourth transmitting member 240. In the illustrated embodiment, the transmitting members 210, 220, 230, and 240 are light fibers. The light fibers are flexible and include a curved portion at the distal end portions to direct the light toward bodily tissue disposed adjacent the housing of the probe portion 202 of the device 200.

The first transmitting member 210 is configured to receive light or light waves from an electromagnetic radiation source and transmit the light to a location within the body of the patient. More specifically, the first transmitting member 210 is configured to transmit the light to a location within the body of the patient and direct the light towards or into bodily tissue of the patient. In the illustrated embodiment, a distal end portion of the first transmitting member is bent or curved and the distal tip is pointed such that the light escapes or exits the first transmitting member 210 from the distal tip and is directed toward the bodily tissue to be observed.

In some embodiments, the first electromagnetic radiation source is configured to produce or generate light waves and direct the light waves to the first transmitting member 210. In some embodiments, the first electromagnetic radiation source is a lamp or other type of light generation device. In some embodiments, the first electromagnetic radiation source is configured to produce or generate light within a range of specific wavelengths. For example, in some embodiments, the first electromagnetic radiation source is a pulsed xenon arc lamp and is configured to produce light with wavelengths between 320 to 920 nm or is configured to product a spectrum of electromagnetic radiation centered about (or focused around) a particular wavelength (such as 500 nm). In other embodiments, the first electromagnetic radiation source is a different type of bulb and is configured to produce light with a different range of wavelengths.

The second transmitting member 220 includes a first end portion and a second end portion 224. The second transmitting member is configured to transmit light from its second end portion to its first end portion. The second transmitting member is configured to receive light or light waves from within the body of the patient and transmit such light or light waves from a location within the body of the patient to a location outside of the body of the patient. For example, in the illustrated embodiment, the second transmitting member is configured to receive light of the first electromagnetic radiation source that has been delivered to bodily tissue of the patient via the first transmitting member 210 and reflected or scattered by the bodily tissue. Said another way, light or light waves of the first source are transmitted from the first electromagnetic radiation source to the bodily tissue via the first transmitting member 210. The light or light waves or some portion of the light or light waves may be scattered or reflected by the bodily tissue. The light or light waves that are scattered, such as reflected or emitted, by the bodily tissue is received by the distal end portion 224 of the second transmitting member 220.

The first end portion of the second transmitting member 220 is operatively coupled to a first electromagnetic radiation detector. Once the light or light waves are received by the second end portion 224 of the second transmitting member 220, the light or light waves are transmitted to the first end portion of the second transmitting member 220 and delivered to the first electromagnetic radiation detector.

The first electromagnetic radiation detector is configured to receive the light or the light waves and provide data for analysis. For example, in some embodiments, the first electromagnetic radiation detector is configured to output intensity data or a spectrum of the light or light waves received by the second transmitting member 220. In some embodiments, the first electromagnetic radiation detector is configured to deliver the output to a computing device, which is configured to analyze the output. In some embodiments, the computing device is configured to analyze the output according to a first modality, such as an angle-resolved low coherence interferometry (aLCI) or another modality.

The third transmitting member 230 includes a first end portion and a second end portion 234. In some embodiments, the first end portion is configured to be disposed outside of a body of a patient while the second end portion 234 is configured to be disposed within the body of the patient. The first end portion is operatively coupled to a second electromagnetic radiation source. As described in further detail below, the second electromagnetic radiation source is configured to generate and direct light (such as light of varying wavelengths) into the third transmitting member 230. In some embodiments, the second electromagnetic radiation source is different than the first electromagnetic radiation source. For example, in some embodiments, the first electromagnetic radiation source is of a first type and the second electromagnetic radiation source is of a second, different type.

The third transmitting member 230 is configured to receive the light from the second electromagnetic radiation source and transmit the light to a location within the body of the patient. More specifically, the third transmitting member 230 is configured to transmit the light to a location within the body of the patient and direct the light towards or into bodily tissue of the patient. For example, in some embodiments, the third transmitting member 230 is configured to transmit the light of the second electromagnetic radiation source such that the light escapes or exits the third transmitting member 230 from the distal or second end portion 234 of the third light transmitter.

In some embodiments, the second electromagnetic radiation source is configured to produce or generate light waves and direct the light waves to the third transmitting member 230. In some embodiments, the second electromagnetic radiation source is a diode laser or another type of light generation device. In some embodiments, the second electromagnetic radiation source is configured to produce or generate light within a range of specific wavelengths. For example, in some embodiments, the second electromagnetic radiation source is configured to produce light with wavelengths between about 5,000 and 13,000 nm. In other embodiments, the second electromagnetic radiation source is a configured to produce light with a different range of wavelengths.

The fourth transmitting member 240 includes a first end portion and a second end portion 244. The fourth transmitting member 240 is configured to transmit light from its second end portion 244 to its first end portion. The fourth transmitting member 240 is configured to receive light or light waves from within the body of the patient and transmit such light or light waves from a location within the body of the patient to a location outside of the body of the patient. For example, in some embodiments, the fourth transmitting member 240 is configured to receive light of the second electromagnetic radiation source that has been delivered to bodily tissue of the patient via the third transmitting member 230 and reflected or scattered by the bodily tissue. Said another way, light or light waves of the second electromagnetic radiation source are transmitted from the second electromagnetic radiation source to the bodily tissue via the third transmitting member 230. The light or light waves or some portion of the light or light waves may be scattered or reflected by the bodily tissue. The light or light waves that are scattered or reflected by the bodily tissue is received by the distal end portion 244 of the fourth transmitting member 240.

The first end portion of the fourth transmitting member 240 is operatively coupled to a second electromagnetic radiation detector. Once the light or light waves are received by the second end portion 244 of the fourth transmitting member 240, the light or light waves are transmitted to the first end portion of the fourth transmitting member 240 and delivered to the second electromagnetic radiation detector.

The second electromagnetic radiation detector is configured to receive the light or the light waves and provide data for analysis. For example, in some embodiments, the second electromagnetic radiation detector is configured to output intensity data or a spectrum of the light or light waves received by the fourth transmitting member 240. In some embodiments, the second electromagnetic radiation detector is configured to deliver the output to the computing device, which is configured to analyze the output. In some embodiments, the computing device is configured to analyze the output according to a second modality, different than the first modality. For example, in some embodiments, according to a Raman scattering approach.

In the illustrated embodiment, the housing 203 defines openings or windows 206 and 208. The electromagnetic radiation transmitted to the bodily tissue by the first transmitting member 210 or the third transmitting member 230 may pass through one of the windows 206 and 208. Also, the light that is received from the tissue by the second transmitting member 220 and 240 may also pass through the windows 206 or 208. In other embodiments, the housing 203 is formulated of an optically clear material. In such embodiments, the electromagnetic radiation that is transmitted to the bodily tissue or received from the bodily tissue may be transmitted uninterrupted through the optically clear material.

In the illustrated embodiment, the windows or openings 206 and 208 are on opposite sides of the housing 203 and the different imaging modalities are configured to receive data associated with bodily tissue that is disposed on opposite sides of the housing 203. In such an embodiment, the physician may rotate the probe portion or housing 203 so that each portion of the bodily tissue is imaged by both imaging modalities. In some embodiments, the medical device includes an indicator that provides an indication as the orientation of the probe or housing 203 while it is in the body of the patient.

In some embodiments, the windows or openings 206 and 208 defined by the housing includes a cover, such as a transparent cover (or a cover that does not interfere with the transmission of the electromagnetic radiation there through). In other embodiment, the windows or openings 206 and 208 do not include any cover.

Although the illustrated embodiment illustrates the first transmitting member 210, the second transmitting member 220, the third transmitting member 230, and the fourth transmitting member 240 as each being single filaments or fibers, in some embodiments the transmitting members may each be a plurality or a bundle of filaments or fibers.

FIG. 4 is a cross-sectional view of a portion of a medical device 300 according to another embodiment. The medical device 300 includes a probe portion 302 that includes a housing 303. The housing 303 defines a lumen or cavity that is configured to house at least a portion of each of a first transmitting member 310, a second transmitting member 320, a third transmitting member 330, and a fourth transmitting member 340. The housing 303 is also configured to house a first reflecting member 398 and a second reflecting member 399.

The first reflecting member 398 and the second reflecting member 399 are configured to receive and direct electromagnetic radiation from the transmitting members and towards the bodily tissue or from the bodily tissue and towards the light transmitters. Specifically, in the illustrated embodiment, the transmitting members 310, 320, 330, and 340 are configured to extend linearly or substantially linearly within the housing 303. The first and second reflecting members 398 and 399 are configured to direct the electromagnetic radiation to the desired locations.

The first reflecting member 398 is configured to direct electromagnetic radiation from the first transmitting member 310 towards bodily tissue (through the window 306), such as along arrow A. The first reflecting member 398 is also configured to direct electromagnetic radiation from the bodily tissue toward the second transmitting member 320, such as along arrow B. In the illustrated embodiment, the first reflecting member 398 is configured to reflect the electromagnetic radiation at an angle of 90 degrees. In other embodiments, the first reflecting member 398 is configured to reflect the electromagnetic radiation at a different angle (such as an acute or obtuse angle).

Similarly, the second reflecting member 399 is configured to direct electromagnetic radiation from the third transmitting member 330 towards bodily tissue (through the window 308), such as along arrow C. The second reflecting member 399 is also configured to direct electromagnetic radiation from the bodily tissue toward the fourth transmitting member 340. In the illustrated embodiment, the second reflecting member 399 is configured to reflect the electromagnetic radiation at an angle of 90 degrees. In other embodiments, the second reflecting member 399 is configured to reflect the electromagnetic radiation at a different angle (such as an acute or obtuse angle).

In some embodiments, the first and second reflecting members 398 and 399 and the transmitting members may be fixedly coupled within the housing 303. In such embodiments, the transmitting members are configured to remain stationary with respect to the first and second reflecting members 398 and 399. For example, a coupling member or an adhesive may be used to fixedly couple the first and second reflecting members 398 and 399 and the transmitting members to the housing 303. In some embodiments, the reflecting members 398 and 399 are movably coupled to the housing 303. In such embodiments, the reflecting members 398 and 399 may be moved to adjust the angle to allow for better transmission or reception of the electromagnetic radiation.

In some embodiments, the first and second reflecting members 398 and 399 are mirrors. In other embodiments, the reflecting members 398 and 399 are other types of reflecting members that are configured to reflect electromagnetic radiation, such as prisms.

In some embodiments, the housing 303 may includes additional members such as optical members, such as lenses or other optical members, configured to focus or direct the electromagnetic radiation towards or away from the transmitting members 310, 320, 330, and 340. The optical members may be disposed between the transmitting members 310, 320, 330, and 340 and the reflecting members 398 and 399. In other embodiments, the optical members are disposed between the reflecting members 398 and 399 and the bodily tissue.

In the illustrated embodiment, the windows or openings 306 and 308 are on opposite sides of the housing 303 and the different imaging modalities are configured to receive data associated with bodily tissue that is disposed on opposite sides of the housing 303. In such an embodiment, the physician may rotate the probe portion or housing 303 so that each portion of the bodily tissue is imaged by both imaging modalities. In some embodiments, the medical device 300 includes an indicator that provides an indication as the orientation of the probe or housing 303 while it is in the body of the patient.

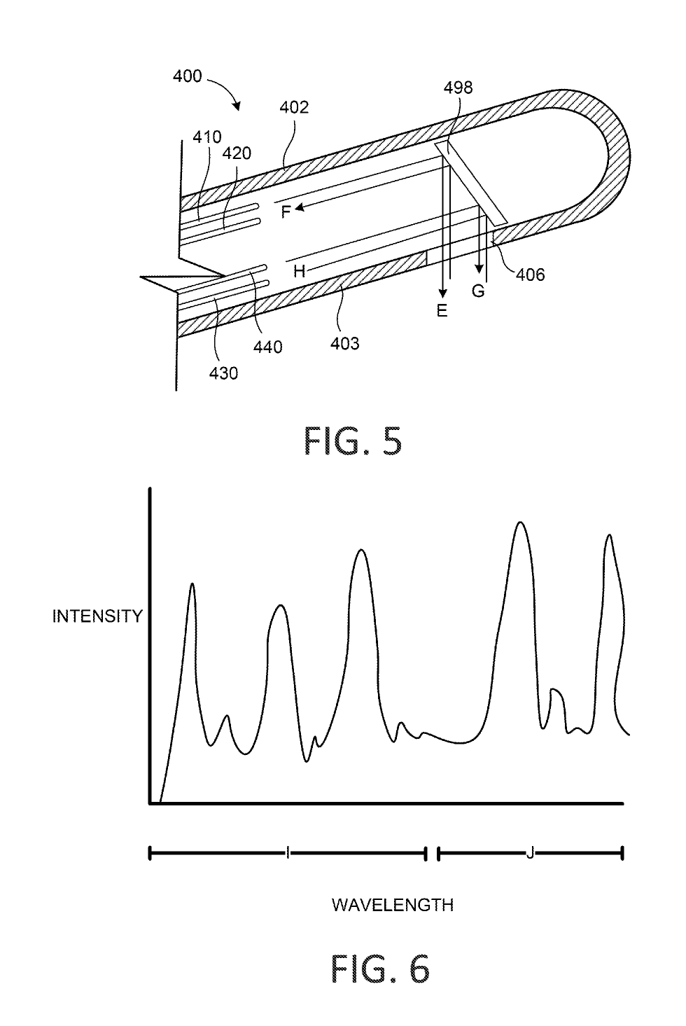

FIG. 5 is a cross-sectional view of a portion of a medical device 400 according to another embodiment. The medical device 400 includes a probe portion 402 that includes a housing 403. The housing 403 defines a lumen or cavity that is configured to house at least a portion of each of a first transmitting member 410, a second transmitting member 420, a third transmitting member 430, and a fourth transmitting member 440. The housing 403 is also configured to house a reflecting member 498.

The reflecting member 498 is configured to receive and direct electromagnetic radiation from the transmitting members and towards the bodily tissue or from the bodily tissue and towards the light transmitters. Specifically, in the illustrated embodiment, the transmitting members 410, 420, 430, and 440 are configured to extend linearly or substantially linearly within the housing 403. The reflecting member 498 is configured to direct the electromagnetic radiation to the desired locations.

The reflecting member 498 is configured to direct electromagnetic radiation from the first transmitting member 410 towards bodily tissue (through the window 406), such as along arrow E. The reflecting member 498 is also configured to direct electromagnetic radiation from the bodily tissue toward the second transmitting member 420, such as along arrow F. Similarly, the reflecting member 498 is configured to direct electromagnetic radiation from the third transmitting member 430 towards bodily tissue (through the window 408), such as along arrow G. The reflecting member 498 is also configured to direct electromagnetic radiation from the bodily tissue toward the fourth transmitting member 440, such as along arrow H.

In the illustrated embodiment, the reflecting member 498 is configured to reflect the electromagnetic radiation at an angle of 90 degrees. In other embodiments, the reflecting member 498 is configured to reflect the electromagnetic radiation at a different angle (such as an acute or obtuse angle).

In some embodiments, the reflecting member 498 and the transmitting members may be fixedly coupled within the housing 403. In such embodiments, the transmitting members are configured to remain stationary with respect to the reflecting member 498. For example, a coupling member or an adhesive may be used to fixedly couple the reflecting member 498 and the transmitting members to the housing 403.

In the illustrated embodiment, the housing defines a single window through which the electromagnetic radiation of both modalities passes through. According, the same bodily tissue may be observed or imaged using both modalities without rotating the housing 403 or the probe portion 402 within the body of the patient.

In some embodiments, the computer or computing device 190 is configured to analyze the data associated with the two modalities of analysis. For example, in some embodiments, the computer or computing device 190 is configured to analyze the data received from the electromagnetic radiation detectors 170 and 190 to provide a diagnosis of the tested or observed bodily tissue.

In some embodiments, the computing device 190 includes a processor and is configured to run programs, such as software programs, that are configured to analyze the data received from the electromagnetic radiation detectors 170 and 190. In some embodiments, the computing device 190 includes a display screen that is configured to display the output or the diagnosis of the tested or observed bodily tissue. In other embodiments, the computing device 190 is configured to provide a print out (such as via a printer) that provides the output or diagnosis of the tested or observed bodily tissue.

In some embodiments, the data received by the computing device 190 from the electromagnetic radiation detectors 160 and 180 is wavelength and intensity data of the electromagnetic radiation that is scattered by the observed bodily tissue. Such data may be assembled to form a fingerprint of the observed bodily tissue. FIG. 6 is a sample fingerprint of bodily tissue. The fingerprint includes intensity and wavelength data for the observed bodily tissue.

In embodiments that include more than two modalities for the analysis, the fingerprint can be formed with the information or analysis of all of the different modalities. For example, in some embodiments, a data of a non-optical modality may be combined with data of optical or other modalities to form the fingerprint.

In some embodiments, the fingerprint of the observed bodily tissue includes data of the first modality (for example, as received by the second light transmitter) and the second modality (for example, as received by the fourth light transmitter). As illustrated in FIG. 6, the data of the first modality I may be combined with or concatenated with the data of the second modality J. The individual fingerprints I and J may be autoscaled or normalized before or after they are concatenated into a single fingerprint in order to enable reproducible comparison to a database of fingerprints that have also been autoscaled or normalized. The fingerprint of the observed bodily tissue (data from the first modality combined with data of the second modality) may then be compared against fingerprints of bodily tissue that is known to be healthy and fingerprints of bodily tissue that is known to be diseased or unhealthy (for example, cancerous or precancerous).

In some embodiments, data of a plurality or more than two modalities are concatenated into a single fingerprint for observation and analysis.

In some embodiments, such as embodiments where the electromagnetic radiation of the two modalities are directed towards opposite sides of the probe portion of the device, the fingerprint of the observed bodily tissue includes data of the first modality and data of the second modality (but the device must be rotated within the body such that the bodily tissue that is observed using the first modality is the same bodily tissue that is observed using the second modality). A balloon or other type anchoring device or mechanism, such as an anchoring sheath, may be used to anchor or steer the probe or the device.

In some embodiments, the fingerprint includes different data points or intensities of radiation that is received as the light sources scan through (or produce radiation of different wavelengths) the range of wavelengths associated with that particular light source. In other embodiments, the bodily tissue is exposed to the entire range of wavelengths at once or simultaneously.

In some embodiments, the computer or computing device 190 is configured to compare the fingerprint of the observed bodily tissue with fingerprints of bodily tissue that is known to be healthy and fingerprints of bodily tissue that known to be diseased or unhealthy. In some embodiments, the fingerprints of bodily tissue that is known to be healthy and fingerprints of bodily tissue that is known to be diseased or unhealthy are stored in a database. In some embodiments, the computer or computing device 190 is configured to store or access such database (for example, via a network or the Internet) to compare the fingerprint of the observed bodily tissue with the fingerprints of bodily tissue of known states.

In some embodiments, the computer or computing device 190 is configured to perform an analysis to compare the fingerprint data. For example, in some embodiments, the computer or computing device 190 is configured to use a multivariate analysis, such as a principal components analysis and a linear discriminate analysis, to compare the fingerprint of the observed bodily tissue with the fingerprints of the bodily tissue of known states. In some embodiments, the comparison of the fingerprints (using a multivariate analysis) allows or provides for the diagnosis of the observed bodily tissue. For example, the observed bodily tissue may be diagnosed as healthy (if the fingerprint of the observed bodily tissue resembles the fingerprints of healthy bodily tissue) or unhealthy (if the fingerprint of the observed bodily tissue resembles the fingerprints of unhealthy bodily tissue).

For example, in some embodiments, the most diagnostically significant features of the fingerprints (of the observed bodily tissue and the bodily tissue of known states) are compared to make a determination of the state of the observed bodily tissue.

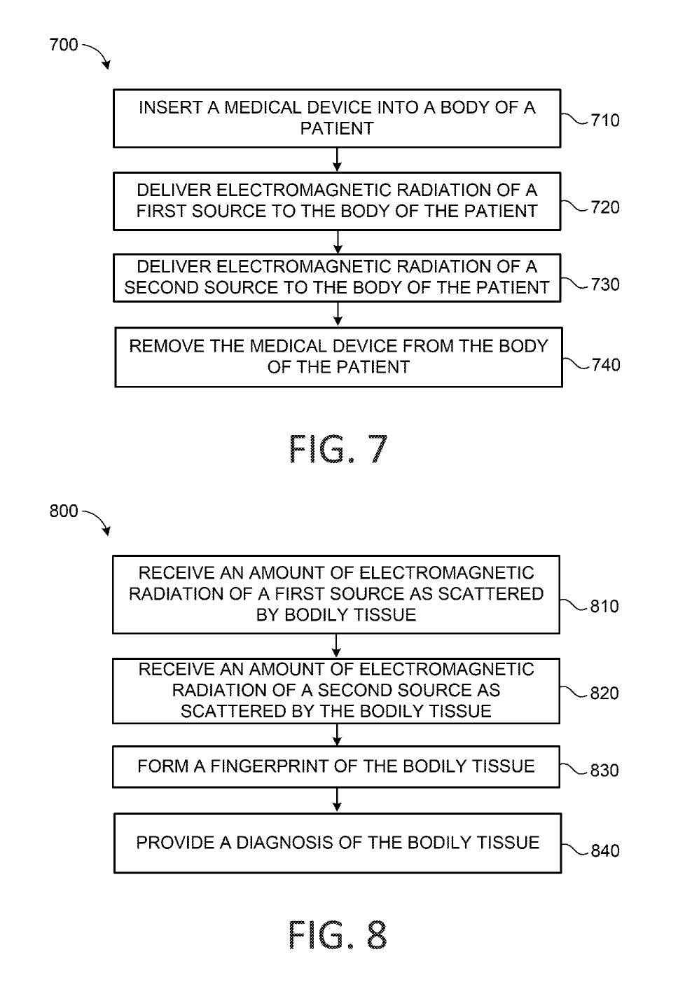

FIG. 7 is a flow chart of a method 700 according to an embodiment of the invention. At 710, a medical device is inserted into a body of a patient. The medical device may be similar to those described above and may be configured to be inserted into a body of a patient and provide a diagnosis of bodily tissue based on a plurality of analysis or modalities.

At 720, electromagnetic radiation is delivered to the body of the patient. The electromagnetic radiation is generated by a first electromagnetic source and delivered to bodily tissue of the patient within the body of the patient. In some embodiments, the medical device includes the first electromagnetic source or the first electromagnetic source is operatively coupled to the medical device. In some embodiments, a single wavelength of radiation is delivered to the bodily tissue at a time. In other embodiments, the bodily tissue is exposed to several different wavelengths of radiation simultaneously.

In some embodiments, the electromagnetic radiation is delivered to the body of the patient via a transmission member such as an optical fiber. In some embodiments, the electromagnetic radiation is delivered to the body of the patient via a transmission member and a reflection member, such as mirror.

In some embodiments, the method includes receiving electromagnetic radiation of the first electromagnetic radiation source that is scattered by the bodily tissue. For example, in some embodiments, the bodily tissue reflects, emits, or otherwise scatters electromagnetic radiation in response to being exposed to the electromagnetic radiation of the first electromagnetic radiation source. In some embodiments, the receipt of the radiation scattered by the bodily tissue is received by a transmission member, such as an optical fiber, and transmitted to an electromagnetic radiation detector.

At 730, electromagnetic radiation of a second electromagnetic source is delivered to the body of the patient. For example, the electromagnetic radiation of the second electromagnetic source may be delivered to the bodily tissue that received the electromagnetic radiation of the first electromagnetic source. In some embodiments, the first electromagnetic source is different than the second electromagnetic source. In some embodiments, the medical device includes the second electromagnetic source or the second electromagnetic source is operatively coupled to the medical device. In some embodiments, a single wavelength of radiation is delivered to the bodily tissue at a time. In other embodiments, the bodily tissue is exposed to several different wavelengths of radiation simultaneously.

In some embodiments, the electromagnetic radiation of the second source is delivered to the body of the patient via a transmission member such as an optical fiber. In some embodiments, the electromagnetic radiation is delivered to the body of the patient via a transmission member and a reflection member, such as mirror.

In some embodiments, the method includes receiving electromagnetic radiation of the second electromagnetic radiation source that is scattered by the bodily tissue. For example, in some embodiments, the bodily tissue reflects, emits, or otherwise scatters electromagnetic radiation in response to being exposed to the electromagnetic radiation of the second electromagnetic radiation source. In some embodiments, the receipt of the radiation scattered by the bodily tissue is received by a transmission member, such as an optical fiber, and transmitted to an electromagnetic radiation detector.

In some embodiments, the medical device may be rotated within the body of the patient to observe a different portion of the bodily tissue. In some embodiments, the medical device may be moved to a different location within the body of the patient to observe different bodily tissue of the patient.

At 740, the medical device is removed from the body of the patient. In some embodiments, the medical device is not removed from the body of the patient until after the electromagnetic radiation of the first and second sources are delivered to the body of the patient.

FIG. 8 illustrates a method 800 according to an embodiment of the invention. At 810, an amount to electromagnetic radiation of a first source as scattered by bodily tissue is received.

At 820, an amount of electromagnetic radiation of a second source as scattered by the bodily tissue is received.

At 830, a fingerprint of the bodily tissue is formed. In some embodiments, the fingerprint includes data associated with the electromagnetic radiation of the first source as scattered by the bodily tissue and data associated with electromagnetic radiation of the second source as scattered by the bodily tissue.

In some embodiments, the fingerprint of the observed bodily tissue is compared with fingerprints of bodily tissue of known states.

At 840, a diagnosis of the bodily tissue is provided. For example, in some embodiments, the diagnosis that is provided may be that the observed bodily tissue is healthy or that the observed bodily tissue is unhealthy. In some embodiments, the diagnosis is provided or displayed on a computer screen or other electronic display. In other embodiments, the diagnosis is provided or printed on a physical item, such as a piece of paper.

In some embodiments, the diagnosis of the observed bodily tissue is based on a comparison of the data associated with the observed bodily tissue and the data associated with bodily tissue that is known to be healthy and/or with the data associated with bodily tissue that is known to be unhealthy.

In one embodiment, a medical device, comprising a first transmitting member operatively coupled to a first electromagnetic radiation source and configured to transmit electromagnetic radiation to bodily tissue; a second transmitting member configured to receive electromagnetic radiation from the first electromagnetic radiation source scattered by the bodily tissue; a third transmitting member operatively coupled to a second electromagnetic radiation source and configured to transmit electromagnetic radiation to the bodily tissue, the second electromagnetic radiation source being different than the first electromagnetic radiation source; and a fourth transmitting member configured to receive electromagnetic radiation from the second electromagnetic radiation source scattered by the bodily tissue.

In some embodiments, the first transmitting member is coupled to the second transmitting member and the second transmitting member being operatively coupled to an electromagnetic radiation detector. In some embodiments, the first transmitting member, the second transmitting member, the third transmitting member, and the fourth transmitting member are coupled together.

In some embodiments, the electromagnetic radiation source is configured to produce electromagnetic radiation within a first range of wavelengths and the second electromagnetic radiation source is configured to produce electromagnetic radiation within a second range of wavelengths. The first range of wavelengths is different than the second range of wavelengths.

In some embodiments, the medical device includes a reflecting member configured to direct electromagnetic radiation transmitted by the first transmitting member towards the bodily tissue.

In some embodiments, the medical device includes a reflecting member configured to direct electromagnetic radiation transmitted by the first transmitting member towards the bodily tissue and configured to direct electromagnetic radiation transmitted by the third transmitting member towards the bodily tissue.

In some embodiments, medical device includes a first reflecting member configured to direct electromagnetic radiation transmitted by the first transmitting member towards the bodily tissue; and a second reflecting member configured to direct electromagnetic radiation transmitted by the third transmitting member towards the bodily tissue.

In some embodiments, the medical device includes a housing defining a lumen, at least a portion of the first transmitting member being disposed within the lumen, at least a portion of the second transmitting member being disposed within the lumen, at least a portion of the third transmitting member being disposed within the lumen, at least a portion of the fourth transmitting member being disposed within the lumen.

In some embodiments, the first transmitting member includes a plurality of optical fibers bundled together.

In some embodiments, a method includes inserting a medical device into a body of a patient such that the medical device is disposed adjacent bodily tissue; delivering electromagnetic radiation of a first electromagnetic radiation source to the bodily tissue; delivering electromagnetic radiation of a second electromagnetic radiation source to the bodily tissue, the second electromagnetic radiation source being different than the first electromagnetic radiation source; and removing the medical device from the body of the patient.

In some embodiments, the delivering electromagnetic radiation of a first electromagnetic radiation source includes delivering electromagnetic radiation via an optical fiber.

In some embodiments, the delivering electromagnetic radiation of a first electromagnetic radiation source includes delivering electromagnetic radiation via an optical fiber and the delivering electromagnetic radiation of a second electromagnetic radiation source includes delivering electromagnetic radiation via the optical fiber.

In some embodiments, the delivering electromagnetic radiation of the first electromagnetic radiation source includes delivering electromagnetic radiation via a reflecting member, and the delivering electromagnetic radiation of the second electromagnetic radiation source includes delivering electromagnetic radiation via the reflecting member.

In some embodiments, the delivering electromagnetic radiation of the first electromagnetic radiation source includes delivering electromagnetic radiation via a first reflecting member, and the delivering electromagnetic radiation of the second electromagnetic radiation source includes delivering electromagnetic radiation via a second reflecting member different than the first reflecting member.

In some embodiments, the method includes receiving electromagnetic radiation of the first electromagnetic radiation source scattered by the bodily tissue; transmitting the received electromagnetic radiation to an electromagnetic radiation detector; and receiving electromagnetic radiation of the second electromagnetic radiation source scattered by the bodily tissue.

In some embodiments, the delivering electromagnetic radiation of the first electromagnetic radiation source is delivered via a first optical fiber and the delivering electromagnetic radiation of the second electromagnetic radiation source is delivered via a second optical fiber.

In some embodiments, the delivering electromagnetic radiation of the first electromagnetic radiation source is delivered via a first optical fiber and the delivering electromagnetic radiation of the second electromagnetic radiation source is delivered via a second optical fiber.

In some embodiments, a method includes receiving an amount of electromagnetic radiation of a first electromagnetic radiation source as scattered by bodily tissue; receiving an amount of electromagnetic radiation of a second electromagnetic radiation source as scattered by the bodily tissue, the second electromagnetic radiation source being different than the first electromagnetic radiation source; forming a fingerprint of the bodily tissue that includes data associated with the amount of electromagnetic radiation received from the first electromagnetic radiation source and data associated with the amount of electromagnetic radiation received from the second electromagnetic radiation source; and providing a diagnosis of the bodily tissue based on the fingerprint of the bodily tissue.

In some embodiments, the providing a diagnosis of the bodily tissue includes an indication that the bodily tissue is healthy.

In some embodiments, the providing a diagnosis of the bodily tissue includes an indication that the bodily tissue is unhealthy.

In some embodiments, the method includes comparing the fingerprint of the bodily tissue to fingerprints of bodily tissue that is known to be unhealthy.

In some embodiments, the method includes disposing a first light receiver within a body of a patient configured to receive the amount of light of the first electromagnetic radiation source as scattered by the bodily tissue; and disposing a second light receiver within the body of the patient configured to receive the amount of electromagnetic radiation of the second electromagnetic radiation source as scattered by the bodily tissue, the receiving an amount of electromagnetic radiation from the second electromagnetic radiation source occurs while the first electromagnetic radiation receiver and the second electromagnetic radiation receiver are disposed within the body of the patient.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A medical device, comprising:
a first transmitting member operatively coupled to a first electromagnetic radiation source and configured to transmit electromagnetic radiation to bodily tissue;
a second transmitting member configured to receive electromagnetic radiation from the first electromagnetic radiation source scattered by the bodily tissue;
a third transmitting member operatively coupled to a second electromagnetic radiation source and configured to transmit electromagnetic radiation to the bodily tissue, the second electromagnetic radiation source being different than the first electromagnetic radiation source; and
a fourth transmitting member configured to receive electromagnetic radiation from the second electromagnetic radiation source scattered by the bodily tissue.

2. The medical device of claim 1, wherein the first transmitting member is coupled to the second transmitting member, the second transmitting member being operatively coupled to a electromagnetic radiation detector.

3. The medical device of claim 1, wherein the first transmitting member, the second transmitting member, the third transmitting member, and the fourth transmitting member are coupled together.

4. The medical device of claim 1, wherein the electromagnetic radiation source is configured to produce electromagnetic radiation within a first range of wavelengths, the second electromagnetic radiation source is configured to produce electromagnetic radiation within a second range of wavelengths, the first range of wavelengths being different than the second range of wavelengths.

5. The medical device of claim 1, further comprising:
a reflecting member configured to direct electromagnetic radiation transmitted by the first transmitting member towards the bodily tissue.

6. The medical device of claim 1, further comprising:
a reflecting member configured to direct electromagnetic radiation transmitted by the first transmitting member towards the bodily tissue and configured to direct electromagnetic radiation transmitted by the third transmitting member towards the bodily tissue.

7. The medical device of claim 1, further comprising:
a first reflecting member configured to direct electromagnetic radiation transmitted by the first transmitting member towards the bodily tissue; and
a second reflecting member configured to direct electromagnetic radiation transmitted by the third transmitting member towards the bodily tissue.

8. The medical device of claim 1, further comprising:
a housing defining a lumen, at least a portion of the first transmitting member being disposed within the lumen, at least a portion of the second transmitting member being disposed within the lumen, at least a portion of the third transmitting member being disposed within the lumen, at least a portion of the fourth transmitting member being disposed within the lumen.

9. The medical device of claim 1, wherein the first transmitting member includes a plurality of optical fibers bundled together.

10. A method, comprising:
inserting a medical device into a body of a patient such that the medical device is disposed adjacent bodily tissue;
delivering electromagnetic radiation of a first electromagnetic radiation source to the bodily tissue;
delivering electromagnetic radiation of a second electromagnetic radiation source to the bodily tissue, the second electromagnetic radiation source being different than the first electromagnetic radiation source; and
removing the medical device from the body of the patient.

11. The method of claim 10, wherein the delivering electromagnetic radiation of a first electromagnetic radiation source includes delivering electromagnetic radiation via an optical fiber.

12. The method of claim 10, wherein the delivering electromagnetic radiation of a first electromagnetic radiation source includes delivering electromagnetic radiation via an optical fiber and the delivering electromagnetic radiation of a second electromagnetic radiation source includes delivering electromagnetic radiation via the optical fiber.

13. The method of claim 10, wherein the delivering electromagnetic radiation of the first electromagnetic radiation source includes delivering electromagnetic radiation via a reflecting member, and the delivering electromagnetic radiation of the second electromagnetic radiation source includes delivering electromagnetic radiation via the reflecting member.

14. The method of claim 10, wherein the delivering electromagnetic radiation of the first electromagnetic radiation source includes delivering electromagnetic radiation via a first reflecting member, and the delivering electromagnetic radiation of the second electromagnetic radiation source includes delivering electromagnetic radiation via a second reflecting member different than the first reflecting member.

15. The method of claim 10, further comprising:
receiving electromagnetic radiation of the first electromagnetic radiation source scattered by the bodily tissue;
transmitting the received electromagnetic radiation to an electromagnetic radiation detector; and
receiving electromagnetic radiation of the second electromagnetic radiation source scattered by the bodily tissue.

16. The method of claim 10, wherein the delivering electromagnetic radiation of the first electromagnetic radiation source is delivered via a first optical fiber and the delivering electromagnetic radiation of the second electromagnetic radiation source is delivered via a second optical fiber.

17. The method of claim 10, wherein the delivering electromagnetic radiation of the first electromagnetic radiation source is delivered via a first optical fiber and the delivering electromagnetic radiation of the second electromagnetic radiation source is delivered via a second optical fiber.

18. A method, comprising:
receiving an amount of electromagnetic radiation of a first electromagnetic radiation source as scattered by bodily tissue;
receiving an amount of electromagnetic radiation of a second electromagnetic radiation source as scattered by the bodily tissue, the second electromagnetic radiation source being different than the first electromagnetic radiation source;
forming a fingerprint of the bodily tissue that includes data associated with the amount of electromagnetic radiation received from the first electromagnetic radiation source and data associated with the amount of electromagnetic radiation received from the second electromagnetic radiation source; and
providing a diagnosis of the bodily tissue based on the fingerprint of the bodily tissue.

19. The method of claim 18, wherein the providing a diagnosis of the bodily tissue includes an indication that the bodily tissue is healthy.

20. The method of claim 18, wherein the providing a diagnosis of the bodily tissue includes an indication that the bodily tissue is unhealthy.
